(19) United States
(12) Patent Application Publication
(10) Pub. No.: US 2020/0009157 A1
(43) Pub. Date: Jan. 9, 2020

(54) DIMETHYL SULFOXIDE (DMSO) AND HYDROCORTISONE COMPOSITIONS AND METHODS OF USE

(71) Applicant: John W. Backer, Lexington, KY (US)

(72) Inventor: John W. Backer, Lexington, KY (US)

(21) Appl. No.: 16/489,395

(22) PCT Filed: Jun. 22, 2017
(86) PCT No.: PCT/US17/38650

§ 371 Date: Aug. 28, 2019

Related U.S. Application Data
(60) Provisional application No. 62/464,709, filed on Feb. 28, 2017.

(51) Int. Cl. A61K 31/573; A61K 47/20; A61K 9/00; A61K 47/10

(52) U.S. Cl. CPC A61K 31/573; A61K 47/10; A61K 9/0014; A61K 47/20

(57) ABSTRACT

A method for treating pain and reducing swelling and inflammation in a human patient. The method includes applying to an affected area a therapeutically effective amount of a dimethyl sulfoxide and hydrocortisone solution. The method may include the application step being topically applied. The solution may have a concentration of about 30 percent dimethyl sulfoxide, about 1 percent hydrocortisone, about 1 percent menthol and/or a concentration of about 68 percent filler material.

This application claims the benefit of U.S. Provisional Patent Application Ser. No. 62/464,709, filed on Feb. 28, 2017, the disclosure of which is incorporated herein by reference.

### TECHNICAL FIELD

This disclosure relates generally to therapeutic compositions for treatment of pain and various ailments and, more particularly, to therapeutic compositions containing dimethyl sulfoxide (DMSO) and hydrocortisone for administration to humans for various therapeutic uses, such as treating pain, reducing swelling and inflammation and promoting the healing of tissue, while reducing undesirable side effects.

### BACKGROUND

Dimethyl sulfoxide (DMSO) is an organo-sulfur compound with the chemical formula (CH3)250. It is well known that DMSO penetrates the skin very easily and may be used as a topical analgesic, a vehicle for topical application of pharmaceuticals, an anti-inflammatory, an anti-oxidant and numerous other medicinal, therapeutic, pharmaceutical and non-pharmaceutical uses for humans and other animal subjects. DMSO is also often used in stem cell storage and shipment. Despite these uses, DMSO has certain potential side effects including headaches, adverse skin reactions, such as burning, itching and minor irritation on contact with the skin, noxious odor and foul taste.

Cortisol is a steroid hormone or glucocorticoid, which is naturally released by the body as a reaction to stress and a low level of blood glucose. Hydrocortisone is a name used for cortisol when it is used as medication and can be used to treat people lacking an adequate supply of naturally generated cortisol in their bodies. Hydrocortisone is available with or without a prescription. Low strength preparations (0.5% to 2%) are often used without a prescription for the temporary relief of (1) minor skin irritations, itching, and rashes caused by eczema, insect bites, poison ivy, poison oak, poison sumac, soaps, detergents, cosmetics, and jewelry; (2) itchy anal and rectal areas; and (3) itching and irritation of the scalp. It is also used to relieve the discomfort of mouth sores.

Hydrocortisone may be prescribed to relieve the itching, redness, dryness, crusting, scaling, inflammation, and discomfort of various skin conditions; the inflammation of ulcerative colitis (a condition which causes swelling and sores in the lining of the colon, large intestine and rectum) or proctitis; or the swelling and discomfort of hemorrhoids and other rectal problems. Hydrocortisone comes in many forms, including as an ointment, cream, lotion, liquid, gel, medicated cloth towelette, and spray for use on the skin; foam, suppositories, cream, ointment, and enema for rectal use; and paste for use in the mouth. Hydrocortisone may cause side effects, such as drying or cracking of the skin, acne, itching, burning or change in skin color.

Despite their many benefits, DMSO compositions are often not used by physicians and clinicians in favor of other pharmaceutical compositions to avoid the common side effects associated with DMSO even where DMSO may be the most effective therapeutic or pharmaceutic agent. Accordingly, there is a need for a new DMSO composition that is an effective therapeutic agent for a variety of injuries and ailments, yet the particular dosage of DMSO and other components minimizes the adverse side effects.

### SUMMARY

In accordance with one aspect of this disclosure, a method for treating pain and reducing swelling and inflammation is disclosed. The method includes applying to an affected area a therapeutically effective amount of a dimethyl sulfoxide and hydrocortisone solution. The method may include the application step being topically applied. The solution may have a concentration of about 30 percent dimethyl sulfoxide, about 1 percent hydrocortisone, about 1 percent menthol and/or a concentration of about 68 percent filler material. The filler material may be selected from a group consisting of 2-(2-Ethoxyethoxy)ethanol, hydroxypropyl cellulose (HPC), polyacrylic acid and carbomer. The solution may be applied at a frequency of about one to two times per day.

In yet another aspect of the disclosure, a pharmaceutical composition for treating pain and reducing swelling and inflammation is disclosed. The pharmaceutical composition includes about 30 weight percent dimethyl sulfoxide, about 1 weight percent hydrocortisone and about 69 weight percent non-active ingredients. The non-active ingredients may be selected from the group consisting of carbitol, hydroxypropyl cellulose, polyacrylic acid or carbomer. The non-active ingredients may also include about 1 weight percent menthol.

Another related aspect of this disclosure is a method including administering a composition to a patient. The composition includes dimethylsulfoxide and hydrocortisone. The composition may be administered topically, such as by means of a roll-on tube to an affected area of the patient. The composition may include about 30 weight percent dimethyl sulfoxide, about 1 weight percent hydrocortisone, about 1 weight percent menthol and about 68 weight percent non-active ingredients. The non-active ingredients may be selected from the group consisting of carbitol, hydroxypropyl cellulose, polyacrylic acid or carbomer. The administering step may be performed at least two times per day.

### DETAILED DESCRIPTION

In the following detailed description, specific embodiments in which the invention may be practiced are described. These embodiments are described in sufficient detail to enable those skilled in the art to practice the embodiments. Also, it is to be understood that other embodiments may be utilized and that process or other changes may be made without departing from the scope of the disclosure. The following detailed description is not to be taken in a limiting sense, and the scope of the invention is defined only by the appended claims and their equivalents. In accordance with the disclosure, a DMSO (human form) and hydrocortisone pharmaceutical composition and related method of use is hereinafter described.

The DMSO and hydrocortisone composition is primarily used for relieving pain and swelling. The use of the hydrocortisone in combination with the DMSO provides a greater anti-inflammatory effect than using DMSO alone. The composition may be used to treat the following conditions: (1) speeds healing of wounds, burns, muscle and skeletal injuries; (2) headaches; (3) inflammation; (4) osteoarthritis; (5) rheumatoid arthritis; (6) foot conditions, such as plantar fasciitis, bunions, fungus, etc.; (7) skin conditions; (8) shingles; (9) bladder conditions; (10) poison ivy; (11) eczema; (12) reducing pain and swelling in muscle and skeletal injuries; and treatment of symptoms associated with interstitial cystitis.

While the DMSO and hydrocortisone composition may be applied in various forms, one particular embodiment involves a topical application, such as a roll-on application similar to how a deodorant tube, stick or bar is applied. Namely, the DMSO and hydrocortisone composition may come in a rigid plastic container having a body and lid. The body has a ball or roller at the top portion for easily applying the gel-like composition topically to the skin. The DMSO and hydrocortisone composition may be applied to the area needing treatment multiple times a day topically, such as twice a day. Of course, it should be appreciated that the DMSO and hydrocortisone composition may be applied in other forms and dosages.

As with any multi-purpose pharmaceutical composition, the optimum dosage of DMSO and hydrocortisone may be varied slightly depending on the particular application. In one particular embodiment, the DMSO and hydrocortisone composition is about 1% hydrocortisone, about 30% DMSO and optionally about 1% menthol. Menthol is an organic compound made synthetically or obtained from cornmint, peppermint or other mint oils. Menthol has local anesthetic and counterirritant qualities. Menthol's ability to chemically trigger the cold-sensitive TRPM8 receptors in the skin is responsible for the well-known cooling sensation it provokes when inhaled, eaten, or applied to the skin. Menthol is included in many products for a variety of reasons, including as a topical analgesic, where it is used to relieve minor aches and pains, such as muscle cramps, sprains, headaches and similar conditions.

Again, the active ingredients are hydrocortisone and DMSO and these ingredients are essential to the anti-inflammatory nature of the composition. Furthermore, DMSO acts as a carrier to transport the hydrocortisone. The remaining part (i.e., the remaining about 68%) of the composition is typically filler materials that make up the gel. A number of components may be used in the filler, including 2-(2-Ethoxyethoxy)ethanol, also known under the trade name carbitol (an industrial solvent). Other components that may be used as the filler material include hydroxypropyl cellulose (HPC) (a topical ophthalmic protectant and lubricant) and polyacrylic acid or carbomer (an emulsifying agent in pharmaceuticals).

The foregoing descriptions of various embodiments have been presented for purposes of illustration and description. These descriptions are not intended to be exhaustive or to limit the invention to the precise forms disclosed. The embodiments described provide the best illustration of the inventive principles and their practical applications to thereby enable one of ordinary skill in the art to utilize the disclosure in various embodiments and with various modifications as are suited to the particular use contemplated.

1. A method for treating pain and reducing swelling and inflammation, comprising:
applying to an affected area a therapeutically effective amount of a dimethyl sulfoxide and hydrocortisone solution.

2. The method of claim 1, wherein the application step is topically applied.

3. The method of claim 1, wherein the solution has a concentration of about 30 percent dimethyl sulfoxide.

4. The method of claim 1, wherein the solution has a concentration of about 1 percent hydrocortisone.

5. The method of claim 1, wherein the solution has a concentration of about 1 percent menthol.

6. The method of claim 1, wherein the solution has a concentration of about 68 percent filler material.

7. The method of claim 6, wherein the filler material may be selected from a group consisting of 2-(2-Ethoxyethoxy)ethanol, hydroxypropyl cellulose (HPC), polyacrylic acid and carbomer.

8. The method of claim 1, wherein the solution is applied at a frequency of about one to two times per day.

9. A pharmaceutical composition for treating pain and reducing swelling and inflammation, comprising:
about 30 weight percent dimethyl sulfoxide;
about 1 weight percent hydrocortisone; and
about 69 weight percent non-active ingredients.

10. The composition of claim 9, wherein the non-active ingredients are selected from the group consisting of carbitol, hydroxypropyl cellulose, polyacrylic acid or carbomer.

11. The composition of claim 9, wherein the non-active ingredients include about 1 weight percent menthol.

12. A method, comprising:
administering a composition to a patient, the composition including:
dimethylsulfoxide and hydrocortisone.

13. The method of claim 12, wherein the composition is administered topically.

14. The method of claim 13, wherein the topical administration is by means of a roll-on tube to an affected area of the patient.

15. The method of claim 12, wherein the composition includes about 30 weight percent dimethyl sulfoxide.

16. The method of claim 15, wherein the composition includes about 1 weight percent hydrocortisone.

17. The method of claim 16, wherein the composition includes about 1 weight percent menthol.

18. The method of claim 17, wherein the composition includes about 68 weight percent non-active ingredients.

19. The method of claim 18, wherein the non-active ingredients are selected from the group consisting of carbitol, hydroxypropyl cellulose, polyacrylic acid or carbomer.

20. The method of claim 19, wherein the administering step is performed at least two times per day.
